(19) United States
(12) Patent Application Publication
(10) Pub. No.: US 2024/0404192 A1
(43) Pub. Date: Dec. 5, 2024

(54) ORAL CAVITY IMAGE PROCESSING DEVICE AND ORAL CAVITY IMAGE PROCESSING METHOD

(71) Applicant: MEDIT CORP., Seoul (KR)

(72) Inventors: Seunghee KO, Seoul (KR); Hotaik LEE, Seoul (KR)

(73) Assignee: MEDIT CORP., Seoul (KR)

(21) Appl. No.: 18/698,093

(22) PCT Filed: Oct. 14, 2022
(86) PCT No.: PCT/KR2022/015573

§ 371 Date: Apr. 3, 2024

(30) Foreign Application Priority Data
Oct. 14, 2021 (KR) 10-2021-0136827

(51) Int. Cl. G06T 17/00; A61C 13/34; G06T 19/20

(52) U.S. Cl. CPC G06T 17/00; A61C 13/34; G06T 19/20; G06T 2200/24; G06T 2210/41; G06T 2219/2021

(57) ABSTRACT

Provided are an intraoral image processing device and an intraoral image processing method. The intraoral image processing method includes obtaining scan data with respect to an intraoral cavity comprising an object, obtaining, from the scan data, first shape information of an object tooth of the object, the object tooth being an object of a die model, obtaining second shape information for determining a shape of the die model, and, based on the first shape information and the second shape information, obtaining the die model.

### TECHNICAL FIELD

An embodiment of the disclosure relates to an intraoral image processing device and an intraoral image processing method.

### BACKGROUND ART

Recently, as a method of obtaining intraoral information of a patient, a method of obtaining an intraoral image of a patient through a three-dimensional scanner has been used. By scanning the intraoral cavity of a patient through a three-dimensional scanner, three-dimensional scan data with respect to an object such as a tooth, a gum, a jawbone, etc. may be obtained, and the obtained three-dimensional scan data may be used for dental treatment, orthodontics, prosthetic dentistry, etc.

A three-dimensional teeth model generated through the obtained three-dimensional scan data may show the patient the progress of orthodontic treatment and may become data for identifying a part that is difficult to be directly observed in the intraoral cavity of the patient. According to the treatment method, the three-dimensional teeth model may include a die model. A die model denotes a three-dimensional teeth model separately showing individual teeth, and the die model may be detached from or attached to a base of the three-dimensional teeth model. For example, when a prosthetic operation, such as crowning or lamination, is performed on a prepared tooth, a die model reflecting a shape of the prepared tooth may be detached from the three-dimensional teeth model in order to identify whether a prosthetic appliance fits well. Also, for example, when a die model reflecting a shape of an adjacent tooth arranged next to the prepared tooth is generated, whether or not a prosthetic appliance of the prepared tooth and the adjacent tooth are in contact with each other may be identified.

A die model may be formed by cutting a plastic model. However, a die model that is cut by hand may not be elaborate and user convenience thereof may be decreased, and thus, a new method of forming a die model is required.

### DISCLOSURE

### Technical Problem

An embodiment of the disclosure aims to provide an intraoral image processing device and an intraoral image processing method that are capable of obtaining a three-dimensional die model having shape information of an object tooth by using scan data with respect to an intraoral cavity including an object.

### Technical Solution

An intraoral image processing method according to an embodiment includes obtaining scan data with respect to an intraoral cavity comprising an object, obtaining, from the scan data, first shape information of an object tooth of the object, the object tooth being an object of a die model, obtaining second shape information for determining a shape of the die model, and, based on the first shape information and the second shape information, obtaining the die model.

An intraoral image processing device according to an embodiment includes a display, a memory storing one or more instructions, and a processor configured to execute the one or more instructions stored in the memory to obtain scan data with respect to an intraoral cavity comprising an object, obtain, from the scan data, first shape information of an object tooth of the object, the object tooth being an object of a die model, obtain second shape information for determining a shape of the die model, and, based on the first shape information and the second shape information, obtain the die model.

### Advantageous Effects

According to an intraoral image processing device and an intraoral image processing method according to an embodiment of the disclosure, a three-dimensional die model having shape information of an object tooth may be obtained by using scan data with respect to an intraoral cavity including an object.

### DESCRIPTION OF DRAWINGS

FIG. 1 is a diagram for describing an intraoral image processing system according to an embodiment of the disclosure.

FIG. 2 is a diagram for describing a die model according to an embodiment of the disclosure.

FIG. 3 is a block diagram of an intraoral image processing device according to an embodiment of the disclosure.

FIG. 4 is a flowchart of a method, performed by an intraoral image processing device, of obtaining a die model, according to an embodiment of the disclosure.

FIG. 5 is a diagram for describing a method, performed by an intraoral image processing device, of obtaining a die model, according to an embodiment of the disclosure.

FIGS. 6 and 7 are diagrams for describing an operation, performed by an intraoral image processing device, of obtaining shape information of an object tooth which is an object of a die model, according to an embodiment of the disclosure.

FIG. 8 is a diagram for describing a method, performed by an intraoral image processing device, of obtaining shape information of a die model, according to an embodiment of the disclosure.

FIGS. 9 and 10 are diagrams for describing an operation, performed by an intraoral image processing device, of obtaining shape information of a die model according to a user input, according to an embodiment of the disclosure.

FIG. 11 is a flowchart of a method, performed by an intraoral image processing device, of obtaining shape information of a die model, according to an embodiment of the disclosure.

FIGS. 12A and 12B are diagrams for describing, in detail, shape information of a die model in an intraoral image processing device, according to an embodiment of the disclosure.

FIG. 13 is a diagram for describing, in detail, shape information of a die model in an intraoral image processing device, according to an embodiment of the disclosure.

### MODE FOR INVENTION

In this specification, principles of the present disclosure are described and embodiments are disclosed, in order to clarify the scope of the claims of the present disclosure and clearly convey the present disclosure for one of ordinary skill in the art to implement the present disclosure. The embodiments of the disclosure may be implemented in various forms.

Throughout the specification, like reference numerals refer to like elements. Not all elements of the embodiments are described in this specification, and general aspects in the art or the same aspects of the embodiments are not described. The term “part” or “portion” used in the specification may be implemented as software or hardware, and according to embodiments, a plurality of “units” may be implemented as one unit (element), or one “unit” may include a plurality of units (elements). Hereinafter, by referring to the accompanying drawings, the operating principles and the embodiments of the present disclosure are described.

In this specification, an image may include an image indicating at least one tooth or an intraoral cavity including at least one tooth.

Also, in this specification, an image may include a two-dimensional (2D) image with respect to an object or a three-dimensional (3D) model or a 3D image three-dimensionally representing an object. Also, in this specification, an image may denote data needed to two-dimensionally or three-dimensionally represent an object, for example, raw data, etc. obtained from at least one image sensor. In detail, the raw data is data obtained to generate an image. When an object is scanned by using a 3D scanner, the raw data may be data (for example, 2D data) obtained by at least one image sensor included in the 3D scanner.

In this specification, an “object” may include a tooth, gingiva, at least a portion of an intraoral cavity, a teeth model, and/or an artificial structure (for example, an orthodontic appliance, an implant, an artificial tooth, an orthodontic auxiliary instrument inserted into the intraoral cavity, etc.) which may be inserted into the intraoral cavity. Here, the orthodontic appliance may include at least one of a bracket, an attachment, an orthodontic screw, a lingual orthodontic appliance, and a removable orthodontic-maintenance appliance.

In this specification, a “3D intraoral image” may be formed as a polygonal mesh structure. For example, when 2D data is obtained by using the 3D scanner, the data processing device may calculate coordinates of a plurality of examined surface points by using triangulation. As the surfaces of an object are scanned by using the 3D scanner moving along the surfaces of the object, the amount of scanned data may be increased, and the coordinates of the surfaces points may be accumulated. As a result of obtaining the image, a point cloud of vertexes may be identified to indicate a range of the surfaces. Points on the point cloud may indicate actually measured points on a 3D surface of the object. A surface structure may be approximated as adjacent vertices on the point cloud form a polygonal mesh connected by a line segment. The polygonal mesh may be variously determined, for example, as a triangular mesh, a quadrangular mesh, a pentagonal mesh, etc. A relationship between a polygon and an adjacent polygon of this mesh model may be used to extract the characteristics of a boundary of a tooth, for example, a curvature, a minimum curvature, an edge, a spatial relationship, etc.

Hereinafter, embodiments are described in detail with reference to the drawings.

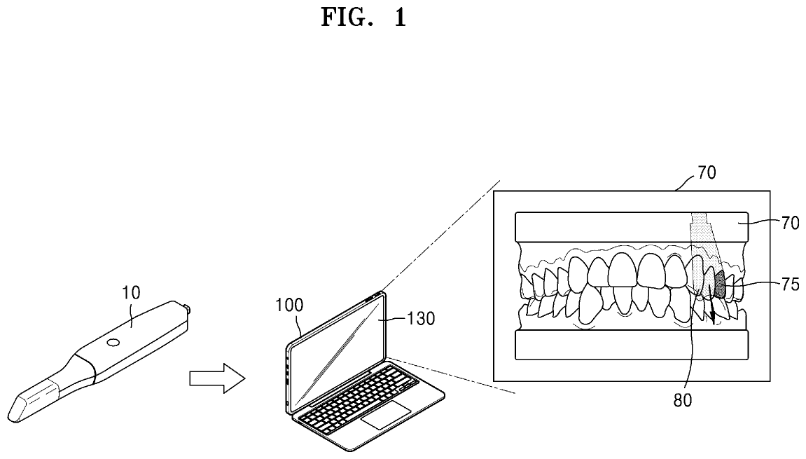

FIG. 1 is a diagram for describing an intraoral image processing system according to an embodiment of the disclosure.

Referring to FIG. 1, the intraoral image processing system may include a 3D scanner 10 and an intraoral image processing device 100.

The 3D scanner 10 according to an embodiment may be a device configured to scan an object and may be a medical device for obtaining an image of an object. The 3D scanner 10 may obtain an image with respect to at least one of an intraoral cavity, an artificial structure, or a plastic model obtained by impression-taking on the intraoral cavity or the artificial structure.

FIG. 1 illustrates the 3D scanner 10 as a type of hand-held scanner which is held by a user in his or her hand and is used to scan an object. However, the 3D scanner 10 is not limited thereto. For example, the 3D scanner 10 may include a type of model scanner, etc. whereby a teeth model is mounted and the mounted teeth model is scanned while moving.

For example, the 3D scanner 10 may be a device inserted into an intraoral cavity and scanning teeth in a non-contact way for obtaining an image with respect to an intraoral cavity including one or more teeth. Also, the 3D scanner 10 may have a form that is possible to be inserted into and withdrawn from an intraoral cavity and may scan an internal state of the intraoral cavity of a patient by using at least one image sensor (for example, an optical camera, etc.).

The 3D scanner 10 may obtain surface information with respect to an object, as raw data, in order to image a surface of at least one of objects including a tooth in the intraoral cavity, gingiva, and an artificial structure (e.g., an orthodontic device including a bracket, a wire, etc., an implant, an artificial tooth, an orthodontic auxiliary instrument inserted into the intraoral cavity, etc.) which may be inserted into the intraoral cavity.

The 3D scanner 10 may transmit the obtained raw data to the intraoral image processing device 100 through a wired or wireless communication network. Image data obtained by the 3D scanner 10 may be transmitted to the intraoral image processing device 100 connected to the 3D scanner 10 through a wired or wireless communication network.

The intraoral image processing device 100 may include all types of electronic devices which are connected to the 3D scanner 10 through a wired or wireless communication network and capable of receiving, from the 3D scanner 10, a 2D image obtained by scanning an object, and generating, processing, displaying, and/or transmitting an image based on the received 2D image.

The intraoral image processing device 100 may include a computing device, such as a smartphone, a laptop computer, a desk top computer, a personal digital assistant (PDA), a tablet personal computer (PC), etc., but is not limited thereto. Also, the intraoral image processing device 100 may also be implemented as a server (or a server device) configured to process an intraoral image.

The intraoral image processing device 100 may generate information by processing the 2D image data received from the 3D scanner 10 or may generate an image by processing the 2D image data. Also, the intraoral image processing device 100 may display the generated information and the generated image through a display 130.

Also, the 3D scanner 10 may directly transmit the raw data obtained through scanning to the intraoral image processing device 100. In this case, the intraoral image processing device 100 may generate, based on the received raw data, a 3D intraoral image three-dimensionally representing the intraoral cavity. The intraoral image processing device 100 according to an embodiment may generate, based on the received raw data, the 3D data (for example, surface data, mesh data, etc.) three-dimensionally representing a shape of the surface of the object.

Also, the “3D intraoral image” may be generated by three-dimensionally modeling an object based on the received raw data, and thus, may also be referred to as a “3D intraoral model.” Hereinafter, models and images two-dimensionally or three-dimensionally representing an object may be collectively referred to as “intraoral images.”

Also, the intraoral image processing device 100 may analyze, process, display, and/or transmit, to an external device, the generated intraoral image.

As another example, the 3D scanner 10 may obtain the raw data through scanning of an object, may generate, by processing the obtained raw data, an image corresponding to the object, and may transmit the generated image to the intraoral image processing device 100. In this case, the intraoral image processing device 100 may analyze, process, display, and/or transmit the received image.

According to an embodiment of the disclosure, the intraoral image processing device 100 may be an electronic device capable of generating and displaying an image three-dimensionally indicating an object, and detailed descriptions thereof are given below.

The intraoral image processing device 100 according to an embodiment may generate a 3D intraoral image (or a 3D intraoral model) by processing received raw data, when the intraoral image processing device 100 receives, from the 3D scanner 10, the raw data obtained by scanning an object. For convenience of explanation, the 3D intraoral image generated by the intraoral image processing device 100 with respect to the object may be referred to as “scan data” hereinafter.

The intraoral image processing device 100 according to an embodiment may obtain a die model 80 corresponding to an object tooth 75 through scan data 70. The intraoral image processing device 100 may display the die model 80 so that the die model 80 is positioned in a base 60. For example, the die model 80 obtained by the intraoral image processing device 100 may be inserted into the base 60 or detached from the base 60. The intraoral image processing device 100 according to an embodiment may include the die model 80 separately detached from the base 60.

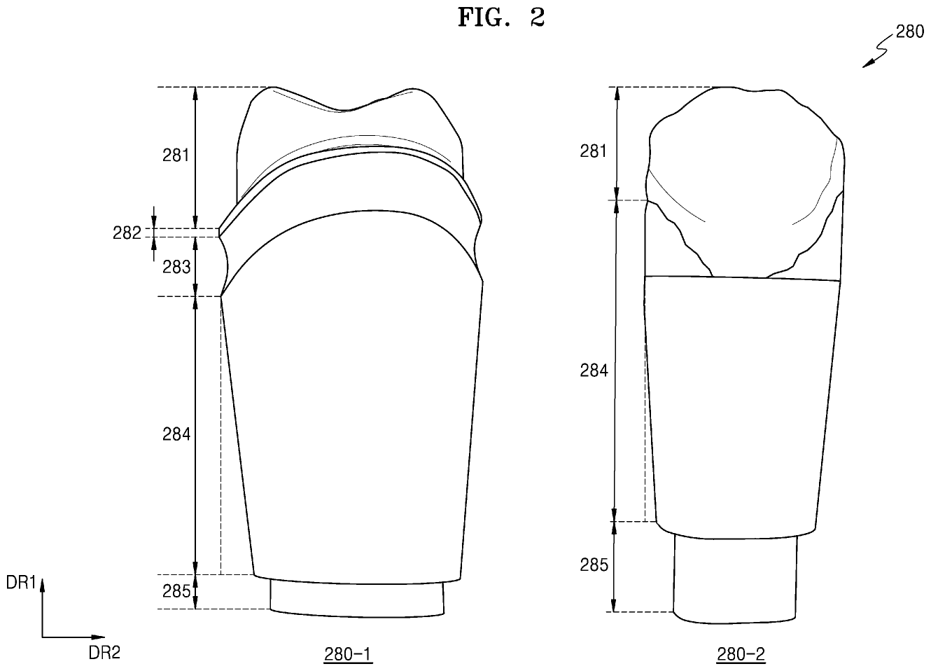

FIG. 2 is a diagram for describing a die model according to an embodiment of the disclosure.

A die model 280 (280-1 and 280-2) according to an embodiment may be a 3D tooth model reflecting a shape of a tooth and may be inserted into a cavity of the base (60 of FIG. 1) or detached from the base 60. The die models 280-1 and 280-2 may have a shape extending in a lengthwise direction DR1 of the die models 280-1 and 280-2, and generally, a length of the die models 280-1 and 280-2 may be greater than a width of the die models 280-1 and 280-2. FIG. 2 illustrates the die model 280-1 reflecting a shape of a prepared tooth and the die model 280-2 reflecting a shape of a pre-preparation tooth. In the present disclosure, the cavity of the base 60 may be a hollow formed in a gingiva area. The prepared tooth may be a tooth, at least a portion of which is removed for a prosthetic operation. The pre-prepared tooth may denote a tooth that is not prepared or may denote an adjacent tooth.

The die models 280-1 and 280-2 may include a tooth shape area 281, a body area 284, and a pin 285. The tooth shape area 281, the body area 284, and the pin 285 may be sequentially positioned in a direction opposite to the lengthwise direction DR1 of the die models 280-1 and 280-2. The tooth shape area 281 may be positioned at an upper portion of the die models 280-1 and 280-2, and the pin 285 may be positioned at a lower portion of the die models 280-1 and 280-2. When the pin 285 is omitted in the die models 280-1 and 280-2, the body area 284 may be positioned at the lower portion of the die models 280-1 and 280-2.

The tooth shape area 281 may be an area reflecting a shape of a tooth. For example, when an object tooth, which is an object of the die models 280-1 and 280-2, is the prepared tooth, the tooth shape area 281 may have the shape of the prepared tooth. For example, when an object tooth, which is an object of the die models 280-1 and 280-2, is the pre-prepared tooth, the tooth shape area 281 may have the shape of the pre-prepared tooth. The tooth shape area 281 may be exposed, when the die models 280-1 and 280-2 are inserted into the cavity of the base 60.

According to an embodiment, the tooth shape area 281 may be obtained based on first shape information of the object tooth, for example, scan data including the object tooth. The tooth shape area 281 may include boundary points of the object tooth based on the scan data.

The body area 284 may form a body of the die models 280-1 and 280-2. Generally, the body area 284 may be inserted into the cavity of the base 60. There may be a certain gap between the body area 284 and the base 60. The certain gap may be set by a user input or may be a predetermined value.

According to an embodiment, the body area 284 may have a tapered shape in which a width of the body area 284 decreases from an upper portion thereof to a lower portion thereof. A side surface of the body area 284 may have a certain inclination with respect to the lengthwise direction DR1. When the die models 280-1 and 280-2 have the tapered shape, the die models 280-1 and 280-2 may be easily inserted into the base 60 and may be easily detached from the base 60.

The pin 285 may be positioned at a lower portion of the die models 280-1 and 280-2, and the die models 280-1 and 280-2 may be fixed to the base 60 through the pin 285. The pin 285 may have a less width than the tooth shape area 281 or the body area 284 but is not limited thereto. Also, the pin 285 may be omitted.

According to an embodiment, the die model 280-2 reflecting the shape of the pre-prepared tooth may include the tooth shape area 281, the body area 284, and the pin 285 described above.

According to an embodiment, the die model 280-1 reflecting the shape of the prepared tooth may include the tooth shape area 281, the body area 284, and the pin 285 described above and may further include a margin line area 282 and a trimming area 283. For example, the tooth shape area 281, the margin line area 282, the trimming area 283, the body area 284, and the pin 285 may be sequentially positioned in a direction opposite to the lengthwise direction DR1 of the die model 280-1.

The margin line area 282 may be an area generated by extending a margin line by a certain length in the lengthwise direction DR1. The margin line may be a boundary line formed by a boundary surface between the object tooth and a prosthetic appliance to be coupled to the object tooth. The margin line may refer to a boundary of the prepared tooth. According to an embodiment, as a length of the margin line area 282 increases, a boundary between the margin line of the die model 280-1 and the prosthetic appliance may come into focus. The margin line area 282 may also be omitted. The length of the margin line area 282 may be set by a user input or may have a pre-set value.

The trimming area 283 may be positioned between the margin line area 282 and the body area 284. The trimming area 283 may be an area inwardly curved in a concave way from the margin line area 282 in a width direction DR2 of the die model 280-1. The trimming area 283 may be trimmed from the margin line area 282 and may make the margin line area 282 stand out. The trimming area 283 may also be omitted. A length of the trimming area 283 or a depth of the trimming area 283 may be set by a user input or may be pre-set.

The intraoral image processing device 100 according to an embodiment may obtain second shape information with respect to a shape of the die models 280-1 and 280-2. For example, the intraoral image processing device 100 according to an embodiment may obtain length information, width information, etc. of areas of each of the die models 280-1 and 280-2 through a user input or a pre-set input. The intraoral image processing device 100 may obtain the die models 280-1 and 280-2 based on the second shape information. For example, the intraoral image processing device 100 may obtain boundary points of a selected object tooth and may obtain polylines based on the boundary points. Here, the boundary points may be points included in a margin line. The intraoral image processing device 100 may obtain the die models 280-1 and 280-2 by generating 3D data (for example, mesh data) based on the polylines. For example, the intraoral image processing device 100 may obtain the margin line area 282, the trimming area 283, the body area 284, and the pin 285 of the die models 280-1 and 280-2 based on the polylines.

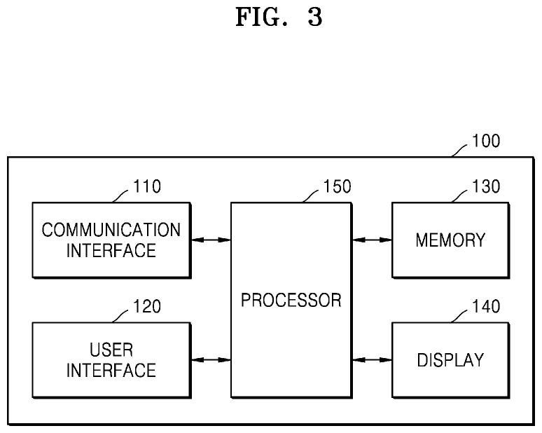

FIG. 3 is a block diagram of an intraoral image processing device according to an embodiment of the disclosure.

Referring to FIG. 3, the intraoral image processing device 100 may include a communication interface 110, a user interface 120, a display 130, a memory 140, and a processor 150.

The communication interface 110 may perform communication with at least one external electronic device (for example, the 3D scanner 10, a server, or an external medical device) through a wired or a wireless communication network. The communication interface 110 may perform communication with at least one external electronic device according to control by the processor 150.

In detail, the communication interface 110 may include at least one short-range wireless communication module performing communication according to the communication standards, such as Bluetooth, Wifi, Bluetooth low energy (BLE), near-field communication (NFC)/radio-frequency identification (RFID), Wifi-direct, ultra-wide band (UWB), or Zigbee.

Also, the communication interface 110 may further include a remote communication module performing communication with a server for supporting remote communication according to the remote communication standards. In detail, the communication interface 110 may include the remote communication module performing communication through a network for Internet communication. Also, the communication interface 110 may include a remote communication module performing communication through a communication network according to the communication standards, such as the 3^{rd} generation (3G), the 4^{th} generation (4G), and/or the 5^{th} generation (5G).

Also, in order to communicate with the external electronic device (for example, the 3D scanner 10, etc.) in a wired manner, the communication interface 110 may include at least one port to be connected to the external electronic device through a wired cable. Accordingly, the communication interface 110 may perform communication with the external electronic device connected in a wired manner thorough the at least one port.

The user interface 120 may receive a user input for controlling the intraoral image processing device 100. The user interface 120 may include a user input device including a touch panel configured to sense a touch of a user, a button configured to receive a push manipulation of the user, a mouse or a keyboard configured to indicate or select a point on a user interface screen, or the like, but is not limited thereto.

Also, the user interface 120 may include a voice recognition device for voice recognition. For example, the voice recognition device may include a microphone, and the voice recognition device may receive a user's voice command or voice request. Accordingly, the processor 150 may control an operation corresponding to the voice command or the voice request to be performed.

The display 130 may display a screen. In detail, the display 130 may display a predetermined screen according to control by the processor 150. In detail, the display 130 may display a user interface screen including an intraoral image generated based on data obtained by the 3D scanner 10 by scanning an intraoral cavity of a patient. Alternatively, the display 130 may display a user interface screen including information related to dental treatment of the patient.

The memory 140 may store one or more instructions. Also, the memory 140 may store one or more instructions executed by the processor 150. Also, the memory 140 may store one or more programs executed by the processor 150. Also, the memory 140 may store data (for example, the raw data, etc. obtained through oral scanning) received from the 3D scanner 10. Alternatively, the memory 140 may store an intraoral image three-dimensionally representing an intraoral cavity.

The processor 150 may execute the one or more instructions stored in the memory 140 to control intended operations to be performed. Here, the one or more instructions may be stored in an internal memory of the processor 150 or in the memory 140 included in the intraoral image processing device separately from the processor 150.

In detail, the processor 150 may execute the one or more instructions to control one or more components included in the intraoral image processing device to perform intended operations. Thus, although the processor is described as performing predetermined operations, it may denote that the processor controls the one or more components included in the intraoral image processing device to perform predetermined operations.

The processor 150 according to an embodiment may execute the one or more instructions stored in the memory 140 to obtain scan data with respect to an intraoral cavity including an object, obtain, from the scan data, first shape information of an object tooth of the object, the object tooth being an object of a die model, obtain second shape information determining a shape of the die model, and, based on the first shape information and the second shape information, obtain the die model.

The processor 150 according to an embodiment may execute the one or more instructions stored in the memory 140 to obtain boundary points of the object tooth, based on the scan data.

The processor 150 according to an embodiment may execute the one or more instructions stored in the memory 140 to display the user interface 120 for setting the second shape information and obtain the second shape information by receiving a user input via the user interface 120 for setting the second shape information.

The processor 150 according to an embodiment may execute the one or more instructions stored in the memory 140 to display the die model so that at least a portion of the die model is positioned in a base. The second shape information may be determined based on at least one of length information of a margin line area of the die model, inner depth information of a trimming area of the die model, whether or not the die model has a pin, a height of a pin of the die model, a gap between the die model and the base, and a direction of the die model.

The processor 150 according to an embodiment may execute the one or more instructions stored in the memory 140 to obtain a first polyline that is apart by a first length from a boundary of the object tooth. The processor 150 may obtain a second polyline including second points, based on first points included in the first polyline. The processor 150 may obtain a bottom line that is apart from the second polyline.

The processor 150 according to an embodiment may execute the one or more instructions stored in the memory 140 to obtain third points, based on the first points. The processor 150 may obtain a third polyline including the third points, positioned between the first polyline and the second polyline in the lengthwise direction of the die model, and positioned to be inward by a first depth from the first polyline in the width direction of the die model. The processor 150 may obtain the die model based on the first polyline, the second polyline, and the third polyline.

The processor 150 according to an embodiment may execute the one or more instructions stored in the memory 140 to obtain crossing points crossing a bottom surface of the base by projecting the points of the second polyline in a direction opposite to the lengthwise direction of the die model. The processor 150 may obtain the bottom line positioned at a position adding the height of the pin to the crossing points in the lengthwise direction of the die model.

The die model may include a body area which is an area from the second polyline to the bottom line. The processor 150 according to an embodiment may execute the one or more instructions stored in the memory 140 to obtain an offset distance between the second polyline and the bottom line, based on an angle formed by a side surface of the body area with respect to the lengthwise direction of the die model and a height of the body area, in the body area which is the area from the second polyline to the bottom line.

The processor 150 according to an embodiment may execute the one or more instructions stored in the memory 140 to obtain the bottom line that is apart from the boundary of the object tooth. The processor 150 may obtain the body area of the die model, which is an area between the boundary and the bottom line.

The die model may include the pin positioned at a lower end portion of the die model. The processor 150 according to an embodiment may execute the one or more instructions stored in the memory 140 to obtain a top line of the pin, removed from the bottom line by a certain width in the width direction of the die model. The processor 150 may obtain the bottom line of the pin apart from the top line of the pin in the direction opposite to the lengthwise direction of the die model by the height of the pin. The processor 150 may obtain the second shape information based on the top line of the pin and the bottom line of the pin.

The processor 150 according to an embodiment may execute the one or more instructions stored in the memory 140 to obtain first vertices that are most adjacent to the boundary of the object tooth from points of the bottom line, respectively, and obtain second vertices that are farthest from the boundary of the object tooth from the points of the bottom line, respectively. The processor 150 may obtain the die model based on the points of the bottom line, the first vertices, and the second vertices.

The processor 150 according to an embodiment may be implemented as a form of processor internally including at least one internal processor and a memory device (for example, random-access memory (RAM), read-only memory (ROM), etc.) for storing at least one of a program, an instruction, a signal, and data to be processed or used by the internal processor.

Also, the processor 150 may include a graphic processing unit (GPU) for processing graphics data corresponding to video data. Also, the processor may be realized as a system on chip (SoC) combining a core and a GPU. Also, the processor may include a multi-core including more cores than a single-core. For example, the processor may include a dual core, a triple core, a quad core, a hexa core, an octa core, a deca core, a dodeca core, a hexadecimal core, or the like.

According to an embodiment of the disclosure, the processor 150 may generate an intraoral image based on a 2D image received from the 3D scanner 10.

In detail, according to control by the processor 150, the communication interface 110 may receive data obtained by the 3D scanner 10, for example, raw data obtained through intraoral scanning. Also, the processor 150 may generate a 3D intraoral image three-dimensionally representing an intraoral cavity, based on the raw data received from the communication interface 110. For example, the 3D scanner 10 may include a camera L corresponding to a left field of view and a camera R corresponding to a right field of view in order to reconstruct a 3D image according to optical triangulation. Also, the 3D scanner 10 may obtain image data L corresponding to the left field of view and image data R corresponding to the right field of view through the camera L and the camera R, respectively. Furthermore, the 3D scanner may transmit the raw data including the image data L and the image data R to the communication interface 110 of the intraoral image processing device 100.

Then, the communication interface 110 may transmit the received raw data to the processor 150, and the processor 150 may generate, based on the received raw data, the intraoral image three-dimensionally representing the intraoral cavity.

Also, the processor 150 may directly receive the intraoral image three-dimensionally representing the intraoral cavity from an external server, a medical device, etc. by controlling the communication interface 110. In this case, the processor may not generate the 3D intraoral image based on the raw data and may obtain the 3D intraoral image.

According to an embodiment of the disclosure, that the processor 150 performs “extraction,” “obtaining,” “generating” operations, etc. may not only denote that the processor 150 directly performs the described operations by executing one or more instructions, but may also denote that the processor 150 controls other components to perform the described operations.

In order to realize one or more embodiments disclosed in this specification, the intraoral image processing device 100 may include only some of the components illustrated in FIG. 3 or may include more components than the components illustrated in FIG. 3.

Also, the intraoral image processing device 100 may store and execute exclusive software synchronized with the 3D scanner 10. Here, the exclusive software may be referred to as an exclusive program, an exclusive tool, or an exclusive application. When the intraoral image processing device 100 operates in synchronization with the 3D scanner 10, the exclusive software stored in the intraoral image processing device 100 may be connected with the 3D scanner 10 and may receive, in real time, pieces of data obtained through intraoral scanning. For example, there may be exclusive software for processing data obtained through intraoral scanning, in the case of the i500 product, which is a 3D scanner of Medit. In detail, Medit manufactures and distributes “Medit link,” which is the software for processing, managing, using, and/or transmitting data obtained by a 3D scanner (for example, 500). Here, the “exclusive software” refers to a program, a tool, or an application operable in synchronization with a 3D scanner, and thus, the “exclusive software” may be shared by various 3D scanners developed and sold by various manufacturers. Also, the described exclusive software may be separately manufactured and distributed from the 3D scanner performing intraoral scanning.

The intraoral image processing device 100 may store and execute the exclusive software corresponding to the i500 product. The exclusive software may perform one or more operations for obtaining, processing, storing, and/or transmitting the intraoral image. Here, the exclusive software may be stored in the processor. Also, the exclusive software may provide a user interface for using the data obtained by the 3D scanner. Here, a screen of the user interface provided by the exclusive software may include the intraoral image generated according to an embodiment of the disclosure.

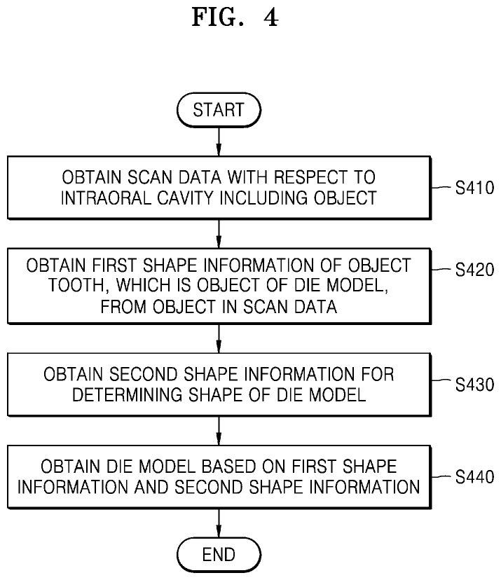

FIG. 4 is a flowchart of a method, performed by an intraoral image processing device, of obtaining a die model, according to an embodiment of the disclosure.

The intraoral image processing method illustrated in FIG. 4 may be performed by the intraoral image processing device 100. Thus, the intraoral image processing method illustrated in FIG. 4 may be a flowchart of operations of the intraoral image processing device 100.

Referring to FIG. 4, in operation S410, the intraoral image processing device 100 may obtain scan data with respect to an intraoral cavity including an object.

The intraoral image processing device 100 according to an embodiment of the present disclosure may receive raw data obtained by scanning an intraoral cavity of a patient or scanning a teeth model via the 3D scanner 10 and may process the received raw data to obtain the scan data with respect to the intraoral cavity including the object. The intraoral image processing device 100 may display the scan data on the display 130.

In operation S420, the intraoral image processing device 100 may obtain, from the scan data, first shape information of an object tooth of the object, the object tooth being an object of a die model.

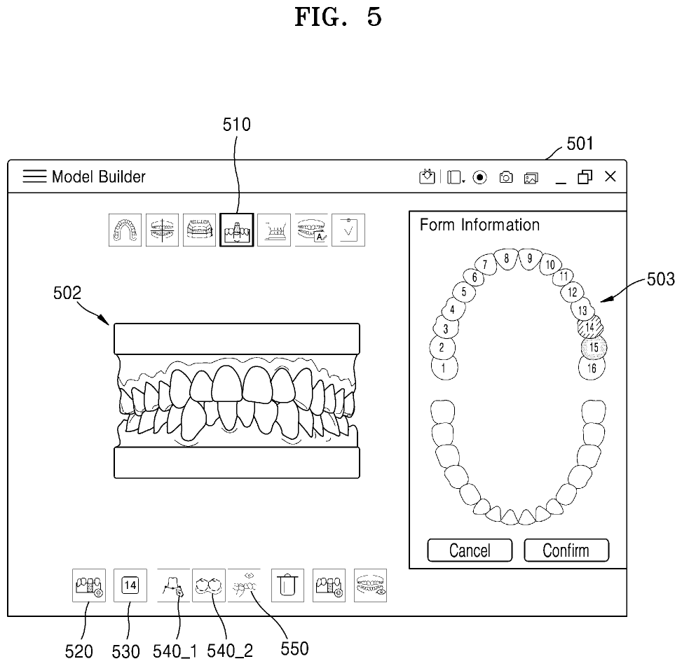

The intraoral image processing device 100 according to an embodiment of the present disclosure may receive a user input for selecting the object tooth through the user interface 120 and, based on the received user input, may determine the object tooth. For example, the intraoral image processing device 100 may display a user interface screen including a die generation icon for generating the die model, based on the scan data. When the user input for selecting the die generation icon is received, the intraoral image processing device 100 may display a screen for selecting the object tooth. When the user input for selecting the object tooth is received, the intraoral image processing device 100 may determine the object tooth for generating the die model, based on the scan data. This aspect will be described in detail with reference to FIG. 5.

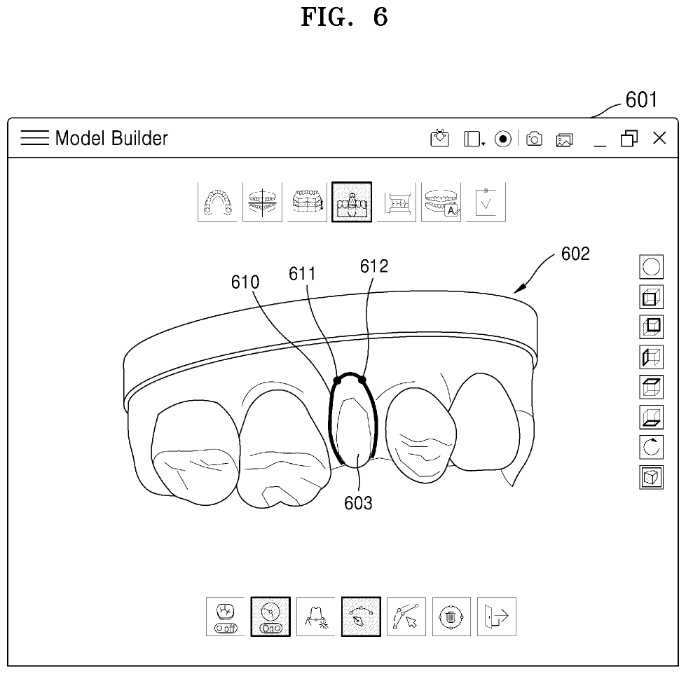

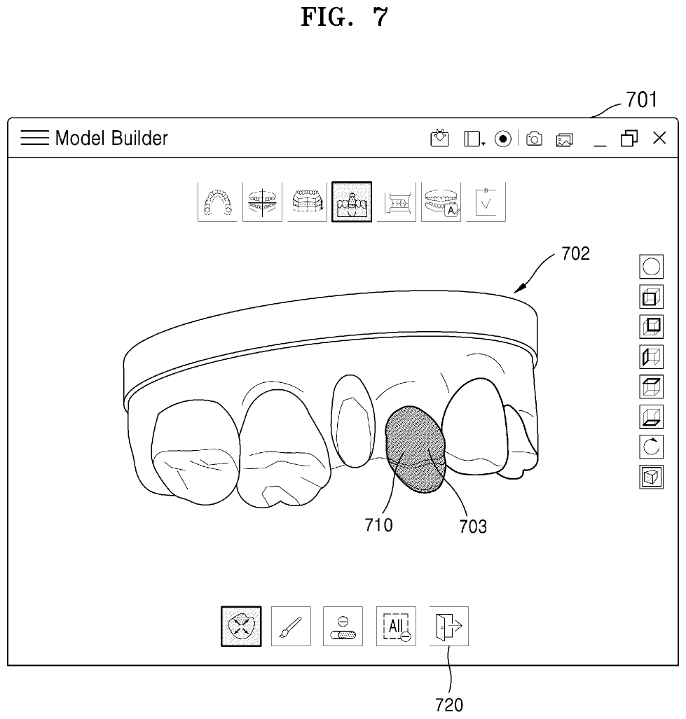

The intraoral image processing device 100 according to an embodiment of the present disclosure may obtain boundary points of the object tooth, based on the scan data. The boundary points may be points included in a margin line. The first shape information may include the boundary points of the object tooth and shape information of the object tooth. For example, the intraoral image processing device 100 may receive a user input for selecting, via the user interface 120, a boundary of the object tooth and may obtain the boundary points of the object tooth based on the received user input. This aspect will be described in detail with reference to FIGS. 6 and 7.

In operation S430, the intraoral image processing device 100 may obtain second shape information for determining a shape of the die model.

The intraoral image processing device 100 according to an embodiment of the present disclosure may obtain the second shape information by receiving a user input with respect to the second shape information through the user interface 120. For example, the intraoral image processing device 100 may display the user interface 120 for setting the second shape information. The intraoral image processing device 100 may obtain the second shape information by receiving a user input for setting, via the user interface 120, the second shape information. The intraoral image processing device 100 according to an embodiment of the present disclosure may obtain the second shape information according to a default setting value of a program.

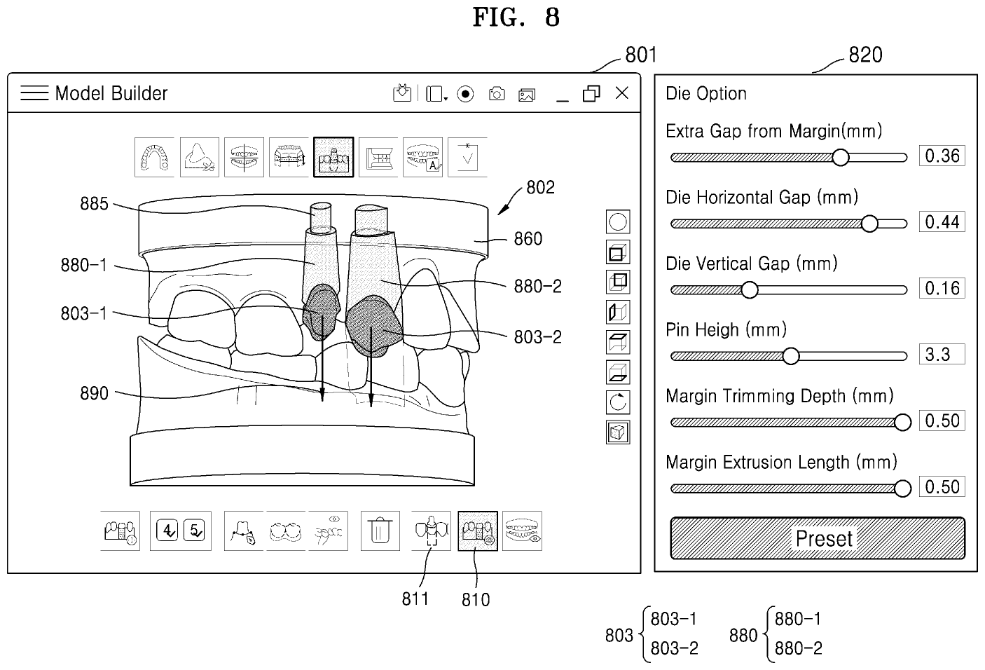

For example, the intraoral image processing device 100 may obtain, through the user input, the second shape information of any one of length information of the margin line area of the die model, inner depth information of a trimming area of the die model, whether or not the die model has a pin, a height of a pin of the die model, a gap between the die model and a base, and a direction of the die model. This aspect will be described in detail with reference to FIGS. 8 to 10.

In operation S440, the intraoral image processing device 100 may obtain the die model, based on the first shape information and the second shape information.

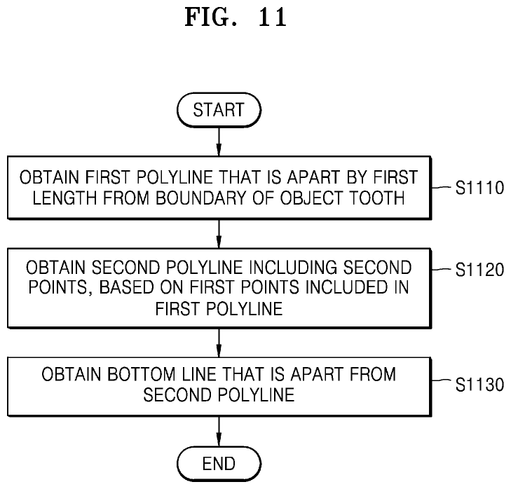

The intraoral image processing device 100 according to an embodiment of the present disclosure may obtain the boundary points based on the first shape information and may obtain polylines based on the boundary points. The intraoral image processing device 100 may obtain the die model by generating 3D data between the boundary points and the polylines. For example, the intraoral image processing device 100 may obtain a tooth shape area (281 of FIG. 2) of the die model based on the first shape information and may obtain a margin line area (282 of FIG. 2), a trimming area (283 of FIG. 2), a body area (284 of FIG. 2), and a pin (285 of FIG. 2) of the die model based on the second shape information. This aspect will be described in detail with reference to FIGS. 11 to 14.

The intraoral image processing device 100 according to an embodiment of the present disclosure may separately obtain the die model reflecting the shape of the object tooth, and thus, user convenience may be improved.

FIG. 5 is a diagram for describing a method, performed by an intraoral image processing device, of obtaining a die model, according to an embodiment of the disclosure.

Referring to FIG. 5, the intraoral image processing device 100 according to an embodiment may generate scan data, based on raw data obtained by the 3D scanner 10. Also, the intraoral image processing device 100 may visually display scan data 502 through a user interface screen 501. The user interface screen 501 may be a screen of the display 130 of FIG. 1. The user interface screen 501 may include one or more menus through which a user may analyze or process the scan data 502.

For example, the user interface screen 501 may include a die generation icon 510 for generating a die model from the scan data 502. When a user input for selecting the die generation icon 510 is received, the intraoral image processing device 100 may display at least one selection device menu. The user interface screen 501 may be a selection device menu and may include a die add menu 520 and an object tooth icon 530. Also, the user interface screen 501 may include an object tooth selection screen 503.

For example, when a user input for selecting the die add menu 520 is received, the intraoral image processing device 100 may display the object tooth selection screen 503. When a user input for selecting one of object teeth of from number 1 to number n (for example, number 32) displayed on the object tooth selection screen 503 is received, the intraoral image processing device 100 may determine an object tooth based on the user input. The intraoral image processing device 100 may display the determined object tooth through the object tooth icon 530. For example, when a user input for selecting the object tooth of number 14 is received through the object tooth selection screen 503, the intraoral image processing device 100 may display the object tooth icon 530 indicating that the object tooth of number 14 is determined.

Also, the user interface screen 501 may include a margin line generation menu 540_1 or an area selection menu 540_2, as a selection device menu for obtaining first shape information of the object tooth. For example, when a user input for selecting the margin line generation menu 540_1 is received, the intraoral image processing device 100 may display a user interface screen 601 as illustrated in FIG. 6.

For example, when a user input for selecting the area selection menu 540_2 is received, the intraoral image processing device 100 may display a user interface screen 701 as illustrated in FIG. 7.

FIGS. 6 and 7 are diagrams for describing an operation, performed by an intraoral image processing device, of obtaining shape information of an object tooth, which is an object of a die model, according to an embodiment of the disclosure.

In FIG. 6, according to a user input for selecting the margin line generation menu 540_1, the intraoral image processing device 100 may display the user interface screen 601 for generating a margin line. The intraoral image processing device 10 may obtain, from scan data 602, a boundary 610 and boundary points (for example, 611 and 612) of an object tooth 603, which is an object of a die model. Here, when the object tooth 603, which is the object of the die model, is a prepared tooth, the boundary 610 of the object tooth 603 may be referred to as a margin line 610. The margin line may include a plurality of points.

For example, based on the scan data with respect to the object tooth 603, the intraoral image processing device 100 may obtain the margin line 610 of the object tooth 603 by receiving a user input for setting the margin line 610 and may display the margin line 610. Also, the intraoral image processing device 100 may detect the points 611 and 612 included in the margin line 610.

In the present disclosure, the method of obtaining the margin line 610 and the points 611 and 612 of the margin line 610 of the object tooth 630 is not limited to the example described above. For example, the intraoral image processing device 100 may automatically recognize the margin line, based on the scan data with respect to the object tooth 603.

In FIG. 7, according to a user input for selecting the area selection menu 540_2, the intraoral image processing device 100 may display the user interface screen 701 for selecting a tooth area 710.

For example, based on a user input for selecting an object tooth 703, the intraoral image processing device 100 may automatically select and display the tooth area 710 of the object tooth 703. The intraoral image processing device 100 may display the tooth area 710 including the object tooth 703 on the user interface screen 701. For example, the intraoral image processing device 100 may implement smart selection and may automatically obtain the tooth area 710 of the object tooth 703. The intraoral image processing device 100 may obtain boundary points (not shown) of the object tooth 703 through the obtained tooth area 710.

While the boundary points of the object tooth 703 obtained through the area selection menu 540_2 may have a less degree of accuracy than the boundary points of the object tooth 603 obtained through the margin line generation menu 540_1, the former boundary points may be more rapidly obtained than the latter boundary points. Although the intraoral image processing device 100 is not limited thereto, the intraoral image processing device 100 may accurately obtain the boundary points (that is, the points of the margin line) through the margin line generation menu 540_1 when the object tooth is a prepared tooth as illustrated in FIG. 6 and may rapidly obtain the boundary points through the area selection menu 540_2 when the object tooth is a pre-prepared tooth as illustrated in FIG. 7.

According to an embodiment, the user interface screen 701 may include an exit menu 720. The intraoral image processing device 100 may determine that the tooth area 710 of the object tooth 703 is obtained, based on a user input for selecting the exit menu 720. When it is determined that tooth area 710 of the object tooth 703 is obtained, the intraoral image processing device 100 may display a die model corresponding to the tooth area, as illustrated in FIG. 8.

FIG. 8 is a diagram for describing a method, performed by an intraoral image processing device, of obtaining shape information of a die model, according to an embodiment of the disclosure.

Referring to FIG. 8, the intraoral image processing device 100 according to an embodiment may obtain second shape information for determining a shape of the die model. The intraoral image processing device 100 according to an embodiment may obtain a die model 880 (880-1 or 880-2) with respect to an object tooth 803 (803-1 or 803-2), based on first shape information and second shape information.

For example, the intraoral image processing device 100 may display a user interface screen 801 for setting the second shape information. The intraoral image processing device 100 may display a die option menu 810, as a selection device menu, on the user interface screen 801. The intraoral image processing device 100 may obtain the second shape information for determining a shape of the die model 880, through the die option menu 810.

For example, when a user input for selecting the die option menu 810 is received, the intraoral image processing device 100 may display a die option screen 820. The die option screen 820 may include a scroll bar for adjusting the second shape information by moving between left and right directions.

For example, the intraoral image processing device 100 may obtain, through the die option screen 820, the second shape information of any one of length information of a margin line area of the die model 880 (“Margin Extrusion Length” in FIG. 8), inner depth information of a trimming area of the die model 880 (“Margin Trimming Depth” in FIG. 8), whether or not the die model 880 has a pin 885, a height of the pin 885 of the die model 880 (“Pin Height” in FIG. 8), a vertical gap between the die model 880 and a base 860 (“Die Vertical Gap” in FIG. 8), a horizontal gap between the die model 880 and the base 860 (“Die Horizontal Gap” in FIG. 8), and a gap between the margin line area and the base 860 of the die model 880 (“Extra Gap from Margin” in FIG. 8). As another example, the intraoral image processing device 100 may further obtain length information of the trimming area of the die model 880, etc.

Also, for example, the intraoral image processing device 100 may include a pin selection menu 811 as a selection device menu and may set whether or not the pin 885 is to be provided through a user input for selecting the pin selection menu. For example, the intraoral image processing device 100 may add the pin 885 or remove the pin 885 through the pin selection menu.

Also, the intraoral image processing device 100 may obtain a direction 890 of the die model 880. For example, the intraoral image processing device 100 may determine the direction 890 of the die model 880 through a user input for dragging an arrow with respect to the direction 890 of the die model 880. Also, for example, the intraoral image processing device 100 may include a die model direction setting menu (for example, 550 of FIG. 5) as a selection device menu. The intraoral image processing device 100 may change the direction 890 of the die model 880 to a direction currently viewed by a user, through a user input for selecting the die model direction setting menu. The method of determining the direction 890 of the die model 880 is not limited to the example described above.

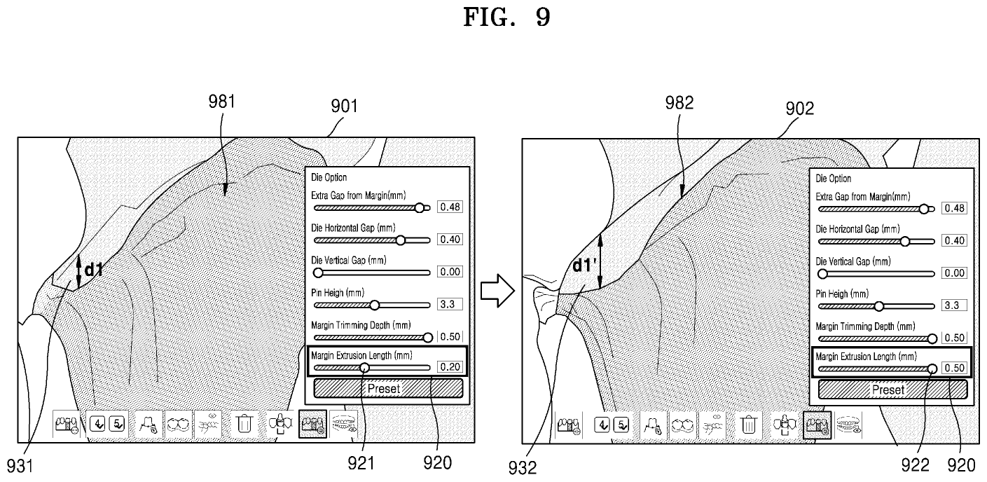

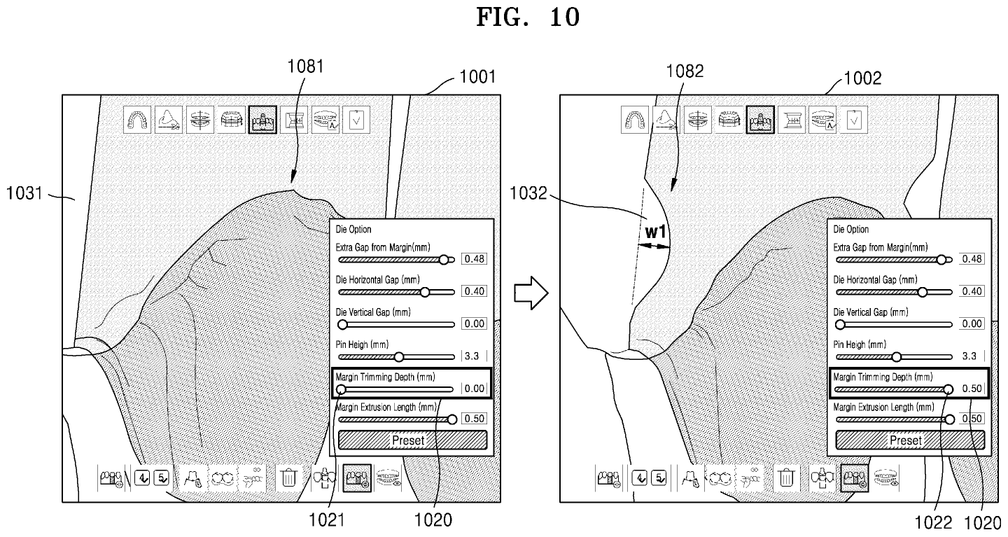

FIGS. 9 and 10 are diagrams for describing an operation, performed by an intraoral image processing device, of obtaining shape information of a die model according to a user input, according to an embodiment of the disclosure.

Referring to FIG. 9, the operation, performed by the intraoral image processing device 100, of obtaining length information of a margin line area of a die model 980, is described.

User interface screens 901 and 902 may include a scroll bar 920 for adjusting a first length d1 of the margin line area. For example, when the scroll bar is moved to the left side, a length of the margin line area may be reduced, and when the scroll bar is moved to the right side, the length of the margin line area may be increased.

When the scroll bar is positioned at a first point 921 on the user interface screen 901, the intraoral image processing device 100 may obtain a margin line area 931 having the first length d1 corresponding to the first point 921. The intraoral image processing device 100 may obtain a die model 981 based on the margin line area 931 having the first length d1.

When the scroll bar is positioned at a second point 922 on the user interface screen 902, the intraoral image processing device 100 may obtain a margin line area 932 having a first length d1′ corresponding to the second point 922. Here, the first length d1′ may be greater than the first length d1. The intraoral image processing device 100 may obtain a die model 982 based on the margin line area 932 having the first length d1′.

Referring to FIG. 10, the operation, performed by the intraoral image processing device 100, of obtaining depth information of a trimming area of a die model 1080, is described.

User interface screens 1001 and 1002 may include a scroll bar 1020 for adjusting an inner depth w1 of the trimming area. For example, when the scroll bar is moved to the left side, a depth of the trimming area may be reduced, and when the scroll bar is moved to the right side, the depth of the trimming area may be increased.

When the scroll is positioned at a first point 1021 on the user interface screen 1001, the intraoral image processing device 100 may obtain a non-inward trimming area 1031. For example, an inner depth of the trimming area 1031 may be 0. The intraoral image processing device 100 may obtain a die model 1081 based on the non-inward trimming area 1031.

When the scroll bar is positioned at a second point 1022 on the user interface screen 1001, the intraoral image processing device 100 may obtain a trimming area 1032 positioned to be inward by a first depth w1. The intraoral image processing device 100 may obtain a die model 1082 based on the trimming area 1032 inward by the first depth w1.

Referring to FIG. 8 again, the intraoral image processing device 100 according to an embodiment may obtain the die model 880 (880-1 or 880-2) with respect to the object tooth 803 (803-1 or 803-2), based on the first shape information and the second shape information. For example, the intraoral image processing device 100 may obtain the die model 880-1 based on the object tooth 803-1 having a shape of a prepared tooth and may obtain the die model 880-2 based on the object tooth 803-2 having a shape of a pre-prepared tooth.

Hereinafter, a detailed method, performed by the intraoral image processing device 100, of obtaining the die model, according to an embodiment of the disclosure, will be described with reference to FIGS. 11 to 13.

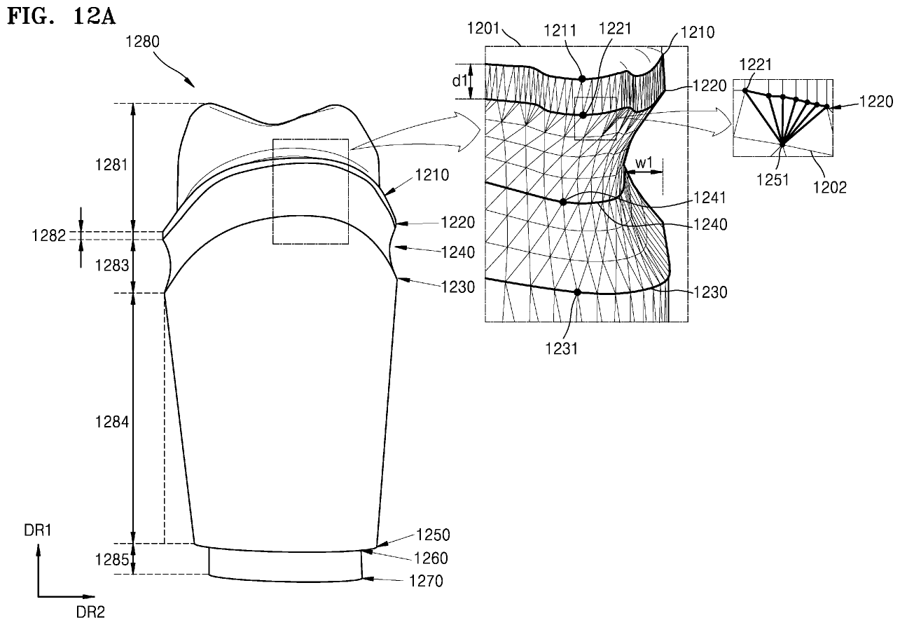

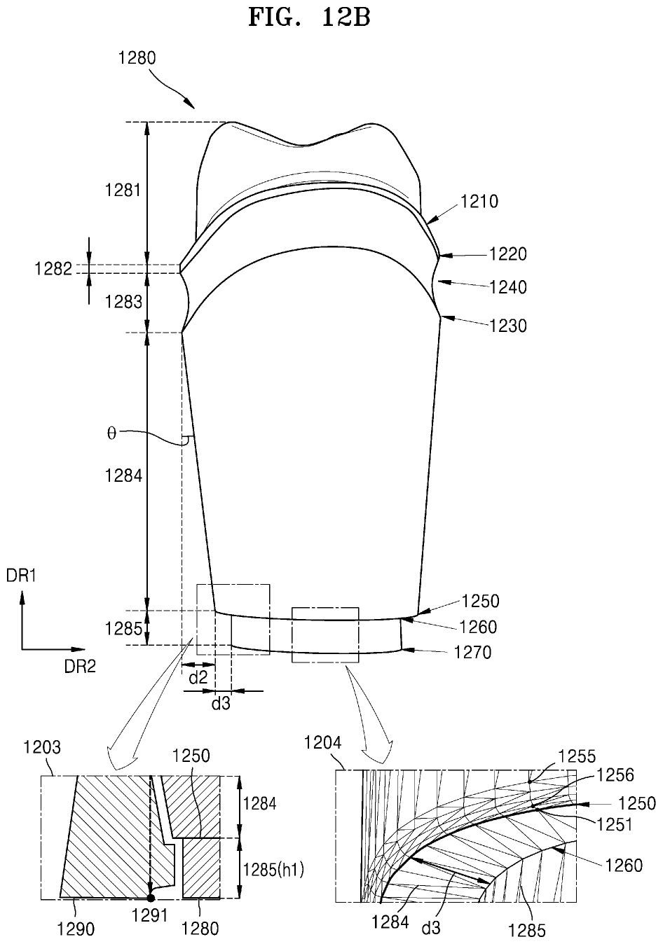

FIG. 11 is a flowchart of a method, performed by an intraoral image processing device, of obtaining shape information of a die model, according to an embodiment of the disclosure. FIGS. 12A and 12B are diagrams for describing, in detail, shape information of the die model in the intraoral image processing device, according to an embodiment of the disclosure.

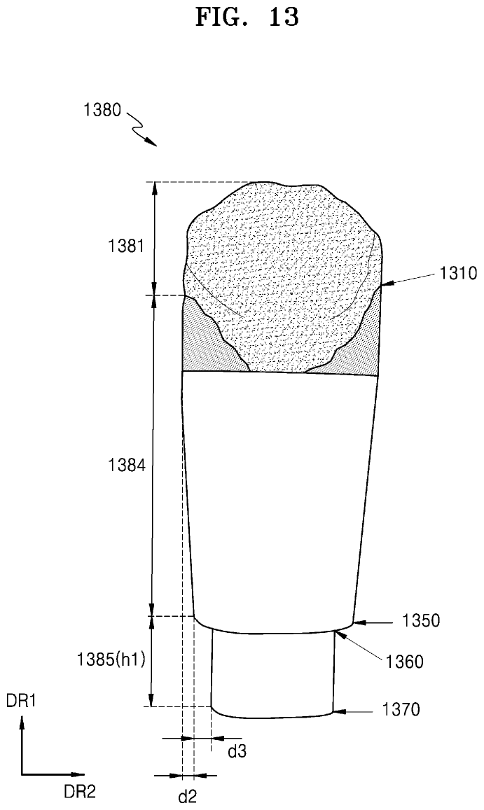

In FIGS. 11, 12A, and 12B, a case where the die model includes a margin line area and a trimming area is illustrated. It will be described with reference to FIG. 13 with respect to a die model not including the margin line area and the trimming area.

Referring to FIG. 11, in operation S1110, the intraoral image processing device 100 may obtain a first polyline that is apart by a first length from a boundary of an object tooth. For example, the intraoral image processing device 100 may obtain the margin line area including an area between the boundary of the object tooth and the first polyline.

Referring to FIG. 12A, when first shape information with respect to a boundary line 1210 of an object tooth is obtained, the intraoral image processing device 100 may obtain a first polyline 1220 apart from the boundary 1210 by a first length d1. When the object tooth is a prepared tooth, the boundary 1210 may be referred to as a margin line. The first polyline 1220 extending from the boundary 1210 by the first length d1 may be referred to as an “extended margin line.” Here, the intraoral image processing device 100 may obtain a margin line area 1282, which is an area between the boundary 1210 of the object tooth and the first polyline 1220.

For example, with reference to an enlarged image 1201, the intraoral image processing device 100 may obtain the first polyline 1220 generated by connecting first points 1221 respectively apart from points 1211 included in the boundary 1210 of the object tooth by the first length d1. Here, the first length d1 may be obtained through a user input for adjusting the scroll bar 910 as illustrated in FIG. 9.

The intraoral image processing device 100 may extend the boundary 1210 of the object tooth to the first polyline 1220 by generating mesh data between the boundary 1210 of the object tooth and the first polyline 1220. For example, the intraoral image processing device 100 may obtain a margin line area 1282 including the mesh data between the boundary 1210 of the object tooth and the first polyline 1220. The margin line area 1282 may have a length corresponding to the first length d1.

In operation S1120, the intraoral image processing device 100 may obtain a second polyline including second points, based on first points included in the first polyline. For example, the intraoral image processing device 100 may obtain a trimming area including an area from the first polyline to the second polyline.

Referring to FIG. 12A, the intraoral image processing device 100 may obtain second points 1231 by sampling the first points 1221 included in a first polyline 1220 and may obtain a second polyline 1230 including the second points 1231. Here, the intraoral image processing device 100 may obtain a trimming area 1283, which is an area between the first polyline 1220 and the second polyline 1230.

Here, the sampling denotes an operation of extracting one point through N adjacent points. For example, with reference to an enlarged image 1202, one point 1251 may be extracted by sampling seven first points (for example, 1221) included in the first polyline 1220. The intraoral image processing device 100 may obtain a polyline including all points (for example, 1251) obtained by sampling all of the first points 1221 included in the first polyline 1220.

Likewise, the intraoral image processing device 100 may obtain the second points 1231 apart from the first points 1221 by a certain distance, by sampling the first points 1221 included in the first polyline 1220. The intraoral image processing device 100 may obtain the second polyline 1230 including the second points 1231 by sampling all of the first points 1221 included in the first polyline 1220. Here, the certain distance may have a pre-set value or a value based on a user input with respect to length information of the trimming area.

Also, the intraoral image processing device 100 may obtain a third polyline 1240 based on the first polyline 1220. For example, the intraoral image processing device 100 may obtain the third polyline 1240 reduced from the first polyline 1220 by offsetting the first polyline 1220.

For example, the intraoral image processing device 100 may obtain the third polyline 1240 positioned between the first polyline 1220 and the second polyline 1230 in a lengthwise direction DR1 of the die model 1280 and inwardly offset from the first polyline 1220 by a first depth w1 in a width direction DR2 of the die model 1280. Third points 1241 of the third polyline 1240 may be central points of the first points 1221 of the first polyline 1220, respectively, and the second points 1231 of the second polyline 1230, respectively, in the lengthwise direction DR1 of the die model 1280. Each of the third points 1241 of the third polyline 1240 may be a point inward from the first points 1221 of the first polyline 1220 by the first depth w1 in the width direction DR2 of the die model 1280. Accordingly, the intraoral image processing device 100 may obtain the trimming area 1283 inwardly curved by the first depth w1 in the width direction DR2 of the die model 1280. Here, the first depth w1 may be obtained through a user input for adjusting the scroll bar 1010 as illustrated in FIG. 10.

The intraoral image processing device 100 may obtain the die model 1280 based on the first polyline 1220, the second polyline 1230, and the third polyline 1240. For example, the intraoral image processing device 100 may generate mesh data between the first polyline 1220, the second polyline 1230, and the third polyline 1240. For example, the intraoral image processing device 100 may generate the mesh data based on a length of the trimming area 1283 in the lengthwise direction DR1 and the number of split pieces of the mesh data.

In operation S1130, the intraoral image processing device 100 may obtain a bottom line that is apart from the second polyline. For example, the intraoral image processing device 100 may obtain a body area including an area from the second polyline to the bottom line.

Referring to FIG. 12B, the intraoral image processing device 100 may obtain a bottom line 1250 apart from the second polyline 1230 by a certain distance. The intraoral image processing device 100 may obtain a body area 1284, which is an area between the second polyline 1230 and the bottom line 1250. Here, the certain distance may be obtained through a user input or pre-set. A height of the body area 1284 may have a value corresponding to the certain distance.

The intraoral image processing device 100 may obtain the body area 1284 having a tapered shape in which a width of the body area 1284 decreases from the second polyline 1230 to the bottom line 1250.

According to an embodiment, the intraoral image processing device 100 may obtain a position of the bottom line 1250 in the lengthwise direction DR1. Referring to an enlarged image 1203, the die model 1280 inserted into a base is illustrated. The intraoral image processing device 100 may obtain crossing points 1291 vertically crossing a bottom surface 1290 of the base by projecting the points of the second polyline 1230 in a direction opposite to the lengthwise direction DR1 of the die model 1280 and may obtain the bottom line 1250 at a position adding a height h1 of the pin 1285 to the crossing points 1291 in the lengthwise direction DR1 of the die model 1280.

According to an embodiment, the intraoral image processing device 100 may obtain a position of the bottom line 1250 in the width direction DR2. For example, the intraoral image processing device 100 may obtain an offset distance d2 between the second polyline 1230 and the bottom line 1250, based on an angle θ formed by a side surface of the body area 1284 with respect to the lengthwise direction DR1 of the die model 1280 and a height of the body area 1284. For example, through a trigonometric formula of tan θ=b/a, the offset distance d2 may be obtained, wherein 8 may be the angle formed by the side surface of the body area 1284 with respect to the lengthwise direction DR1 of the die model 1280, a may be the height of the body area 1284, and b may be the offset distance d2. The offset distance d2 may be calculated based on a minimum height of the body area 1284.

According to an embodiment, referring to an enlarged image 1204, the intraoral image processing device 100 may obtain the die model 1280 having a lower edge having a curvature. For example, the intraoral image processing device 100 may obtain first vertices most adjacent to the boundary 1210 of the die model 1280 from points of the bottom line 1250, respectively, and may obtain second vertices farthest from the boundary 1210 of the die model 1280 from the points of the bottom line 1250, respectively. The intraoral image processing device 100 may obtain the die model 1280 based on the points of the bottom line 1250, the first vertices, and the second vertices.

For example, the intraoral image processing device 100 may obtain a first vertex 1255 most adjacent to the boundary 1210 of the die model 1280 from a point 1251 of the bottom line 1250 and may obtain a second vertex 1256 farthest from the boundary 1210 of the die model 1280 from the 1251 of the bottom line 1250. Based on the point 1251, the first vertex 1255, and the second vertex 1256, the intraoral image processing device 100 may obtain the die model 1280 having the lower edge having the curvature. Here, the second vertex 1256 may be a vertex that is the most adjacent to the pin 1285 from the point 1251 of the bottom line 1250. The intraoral image processing device 100 according to the present disclosure may obtain the die model 1280 which may be easily inserted into the base.

The intraoral image processing device 100 may obtain the body area 1284 by generating mesh data between the second poly line 1230 and the bottom line 1250. For example, the intraoral image processing device 100 may generate the mesh data based on a length between the second poly line 1230 and the bottom line 1250 and the number of split pieces of the mesh data.

The intraoral image processing device 100 may obtain the pin 1285 positioned at a lower portion of the body area 1284. The pin 1285 may be positioned at a lower end portion of the die model 1280. The pin 1285 may include an area between a top line 1260 and a bottom line 1270.

The intraoral image processing device 100 may obtain the top line 1260 of the pin 1285, removed from the bottom line 1250 by a certain width d3 in the width direction DR2. Here, the certain width d3 may be obtained through a user input or pre-set.

The intraoral image processing device 100 may obtain the bottom line 1270 of the pin 1285, apart from the top line 1260 of the pin 1285 by the height h1 of the pin 1285 in a direction opposite to the lengthwise direction DR1 of the die model 1280.

The intraoral image processing device 100 may obtain the die model 1280 including the pin 1285, based on the top line 1260 and the bottom line 1270 of the pin 1285. For example, the intraoral image processing device 100 may generate the pin 1285 by generating mesh data between the top line 1260 of the pin 1285 and the bottom line 1270 of the pin 1285.

FIG. 13 is a diagram for describing, in detail, shape information of a die model in an intraoral image processing device, according to an embodiment of the disclosure. Referring to FIG. 13, a die model 1380 may include a tooth shape area 1381, a body area 1384, and the pin 1285. An object tooth of the die model 1380 may be a pre-prepared tooth but is not limited thereto. The die model 1380 may not include a margin line area and a trimming area.

The intraoral image processing device 100 according to an embodiment of the present disclosure may obtain a bottom line 1350 apart from a boundary 1310 of the object tooth. The intraoral image processing device 100 may obtain a body area 1384, which is an area between the boundary 1310 and the bottom line 1350. In the present disclosure, from among boundaries of the object tooth, the boundary 1310 of the object tooth may be a polyline obtained with respect to a point positioned at a lowermost end with respect to the lengthwise direction DR1, but is not limited thereto.

Similarly to operation S1130, the intraoral image processing device 100 may obtain the body area 1384 having a tapered shape in which a width thereof decreases from the boundary 1310 to the bottom line 1250. The intraoral image processing device 100 may obtain an offset distance d2 between the boundary 1310 and the bottom line 1350.

The intraoral image processing device 100 according to an embodiment of the present disclosure may obtain first vertices most adjacent to the boundary of the object tooth from points of the bottom line 1350, respectively, and may obtain second vertices farthest from the boundary of the object tooth from the points of the bottom line 1350, respectively. The intraoral image processing device 100 may obtain the die model 1380 having a lower edge having a curvature, based on the points of the bottom line 1350, the first vertices, and the second vertices.

The intraoral image processing device 100 according to an embodiment of the present disclosure may obtain a body area 1384 by generating mesh data between the boundary 1310 and the bottom line 1350.

The intraoral image processing device 100 according to an embodiment of the present disclosure may obtain the pin 1385 positioned at a lower portion of the body area 1384.

An intraoral image processing method according to an embodiment of the disclosure may be realized as a program command which may be executed by various computer devices and may be recorded on a computer-readable recording medium. Also, according to an embodiment of the disclosure, a computer-readable storage medium having recorded thereon one or more programs including one or more instructions for executing the intraoral image processing method may be provided.

The computer-readable medium may include a program command, a data file, a data structure, etc. individually or in a combined fashion. Here, examples of the computer-readable storage medium include magnetic media, such as hard discs, floppy discs, and magnetic tapes, optical media, such as compact disc-read only memories (CD-ROMs) and digital versatile discs (DVDs), magneto-optical media, such as floptical discs, and hardware devices configured to store and execute program commands, such as ROMs, RAMs, and flash memories.

Here, a machine-readable storage medium may be provided in a form of a non-transitory storage medium. Here, the “non-transitory storage medium” may denote a tangible storage medium. Also, the “non-transitory storage medium” may include a buffer in which data is temporarily stored.

According to an embodiment, the intraoral image processing method according to various embodiments of the disclosure may be provided by being included in a computer program product. The computer program project may be distributed in the form of a device-readable storage medium (for example, CD-ROM). Alternatively, the computer program may be directly or through online distributed (e.g. download or upload) between two user devices (e.g., smartphones) through an application store (e.g. Play Store, etc.). In detail, the computer program product according to an embodiment of the disclosure may include a storage medium having recorded thereon a program including at least one instruction for executing the intraoral image processing method according to an embodiment of the disclosure.

Although embodiments are described in detail above, the scope of the claims of the present disclosure is not limited thereto, and various modifications and alterations by one of ordinary skill in the art using basic concept of the present disclosure defined by the following claims are also included in the scope of the claims of the present disclosure.

1. An intraoral image processing method comprising:
obtaining scan data with respect to an intraoral cavity comprising an object;
obtaining, from the scan data, first shape information of an object tooth of the object, the object tooth being an object of a die model;
obtaining second shape information for determining a shape of the die model; and
based on the first shape information and the second shape information, obtaining the die model.

2. The intraoral image processing method of claim 1,
wherein the obtaining of the first shape information of the object tooth comprises obtaining boundary points of the object tooth, based on the scan data.

3. The intraoral image processing method of claim 1,
wherein the obtaining of the second shape information for determining the shape of the die model comprises:
displaying a user interface for setting the second shape information; and
obtaining the second shape information by receiving a user input for setting the second shape information via the user interface.

4. The intraoral image processing method of claim 1, further comprising displaying the die model so that at least a portion of the die model is positioned in a base,
wherein the second shape information is determined based on at least one of length information of a margin line area of the die model, inner depth information of a trimming area of the die model, whether or not the die model has a pin, a height of a pin of the die model, a gap between the die model and the base, and a direction of the die model.

5. The intraoral image processing method of claim 1,
wherein the die model comprises a margin line area generated by extending a margin line of the object tooth in a lengthwise direction of the die model.

6. The intraoral image processing method of claim 1,
wherein the die model comprises a body area having a tapered shape in which a width of the body area decreases from an upper portion toward a lower portion of the body area.

7. The intraoral image processing method of claim 5,
wherein the die model further comprises a trimming area positioned at a lower portion of the margin line area and inwardly concave from the margin line area.

8. The intraoral image processing method of claim 1,
wherein the obtaining of the die model, based on the first shape information and the second shape information, comprises:
obtaining a first polyline that is apart by a first length from a boundary of the object tooth;
obtaining a second polyline including second points, based on first points included in the first polyline; and
obtaining a bottom line that is apart from the second polyline.

9. The intraoral image processing method of claim 8,
wherein a pin positioned at a lower portion of the die model is provided, and the obtaining of the die model, based on the first shape information and the second shape information, comprises:
obtaining a top line of the pin, removed from the bottom line by a certain width in a width direction of the die model;
obtaining the bottom line of the pin, apart from the top line of the pin by a height of the pin in a direction opposite to a lengthwise direction of the die model; and
obtaining the die model, based on the top line of the pin and the bottom line of the pin.

10. An intraoral image processing device comprising:
a display;
a memory storing one or more instructions; and
a processor
configured to execute the one or more instructions stored in the memory to:
obtain scan data with respect to an intraoral cavity comprising an object;
obtain, from the scan data, first shape information of an object tooth of the object, the object tooth being an object of a die model;
obtain second shape information for determining a shape of the die model; and
based on the first shape information and the second shape information, obtain the die model.

11. The intraoral image processing device of claim 10,
wherein the processor is further configured to execute the one or more instructions stored in the memory to:
display, on the display, a user interface for setting the second shape information; and
obtain the second shape information by receiving a user input for setting the second shape information via the user interface.

12. The intraoral image processing device of claim 10,
wherein the processor is further configured to execute the one or more instructions stored in the memory to display, on the display, the die model so that at least a portion of the die model is positioned in a base,
wherein the second shape information is determined based on at least one of length information of a margin line area of the die model, inner depth information of a trimming area of the die model, whether or not the die model has a pin, a height of a pin of the die model, a gap between the die model and the base, and a direction of the die model.

13. The intraoral image processing device of claim 10,
wherein the die model comprises a margin line area generated by extending a margin line of the object tooth in a lengthwise direction of the die model.

14. The intraoral image processing device of claim 10,
wherein the die model comprises a body area having a tapered shape in which a width of the body area decreases from an upper portion toward a lower portion of the body area.

15. The intraoral image processing device of claim 10,
wherein
the processor is further configured to execute the one or more instructions stored in the memory to:
obtain a first polyline that is apart by a first length from a boundary of the object tooth;
obtain a second polyline including second points, based on first points included in the first polyline; and
obtain a bottom line that is apart from the second polyline.
